(19) United States
(12) Patent Application Publication
(10) Pub. No.: US 2013/0048883 A1
(43) Pub. Date: Feb. 28, 2013

(54) SYSTEMS AND METHODS FOR PREVENTING UNSAFE MEDICAL TREATMENT

(71) Applicants: William E. Simon, Melbourne, FL (US); Jeffrey A. Simon, Melbourne, FL (US); Michael Bealer, Melbourne, FL (US); Kai Wundke, Indialantic, FL (US); Jeffrey Michael Kapatoes, Viera, FL (US); Matthew J. Daniels, Rockledge, FL (US); Sanjeev Saini, Melbourne, FL (US)

(72) Inventors: William E. Simon, Melbourne, FL (US); Jeffrey A. Simon, Melbourne, FL (US); Michael Bealer, Melbourne, FL (US); Kai Wundke, Indialantic, FL (US); Jeffrey Michael Kapatoes, Viera, FL (US); Matthew J. Daniels, Rockledge, FL (US); Sanjeev Saini, Melbourne, FL (US)

(73) Assignee: SUN NUCLEAR CORPORATION, Melbourne, FL (US)

(21) Appl. No.: 13/601,308

(22) Filed: Aug. 31, 2012

Related U.S. Application Data
(60) Provisional application No. 61/529,571, filed on Aug. 31, 2011.

(51) Int. Cl. A61N 5/00

(52) U.S. Cl. 250/492.3

(57) ABSTRACT

A treatment safety device is operable by a computer-based treatment safety module to prevent, by at least one of an operational interlock and a warning indicator, a medical treatment unless the treatment safety module determines that predetermined treatment verification criteria are met. The treatment safety device can tie into an existing operational interlock, such as an electrical door interlock. In a radiation oncology application, the treatment safety module verification criteria can include a satisfactory correspondence between elements of a pending treatment and an intended treatment.

### CROSS-REFERENCE TO RELATED APPLICATION

This application claims the benefit of U.S. Provisional Application Ser. No. 61/529,571, filed on Aug. 31, 2011, the contents of which are herein incorporated by reference in their entirety.

### FIELD OF INVENTION

The present invention relates to systems and methods for preventing the unsafe administration of potentially hazardous treatments from medical devices, and more particularly, to systems and methods for quality assurance (QA) of radiation therapy beam delivery in clinical oncology.

### BACKGROUND OF THE INVENTION

There is a need for independent process control of radiation oncology (RADONC) treatment to help verify that a treatment about to be delivered to a patient is not unsafe or otherwise inappropriate. This need was echoed by recent RADONC accidents as reported Jan. 23, 2010 in the New York Times (NYT): “The Radiation Boom: Radiation Offers New Cures, and Ways to Do Harm” by Walt Bogdanich. At least two of these accidents resulted in the eventual death of the patient. Quoting from the article—“ . . . its complexity has created new avenues for error—through software flaws, faulty programming, poor safety procedures or inadequate staffing and training. When those errors occur, they can be crippling. ‘Linear accelerators and treatment planning are enormously more complex than 20 years ago,’ said Dr. Howard I. Amols, chief of clinical physics at Memorial Sloan-Kettering Cancer Center in New York. But hospitals, he said, are often too trusting of the new computer systems and software, relying on them as if they had been tested over time, when in fact they have not.”

Some disturbing facts particularly noted in the NYT article:
On 133 occasions, devices used to shape or modulate radiation beams were inadvertently omitted or used improperly, including wedges; and
On 284 occasions, the radiation missed some or all of the intended treatment area, in some cases being directed to the entirely wrong area of the body (e.g., radioactive seeds intended for a man's cancerous prostate were placed in the base of his penis; stomach cancer patient treated for prostate cancer. In 50 instances, this was the result of patients receiving a course of radiation treatment intended for a different patient (e.g., a brain cancer patient received radiation for breast cancer);
In New York State alone, there were 21 accidents relating to beam-modifying devices, leading to a special alert in December 2004 and an additional alert in April 2005, that hospitals need to ensure that radiation field size and shape are appropriate prior to delivery to the patient.
An extremely serious accident occurred in New York in 2005, involving improper and unsafe radiation delivery to a patient, even after these warnings. Among other issues, a radiation therapist incorrectly programmed the computer controlling a linear accelerator (LINAC) to “wedge out” rather than “wedge in” (as the plan required). The medical physics staff failed to notice it during weekly checks, and therapists during subsequent treatments (despite the fact that “wedge OUT” appeared on their screens). On 27 occasions, radiation was delivered with the improper configuration.
Investigations into this accident looked at the computer and software that drove the LINAC. The software required programming instructions be saved in the following order in the computer: a) the quantity or dose of radiation in the beam; b) a digital image of the treatment area; and c) instructions that guide the multileaf collimator. The computer crashed repeatedly and the medical physicist did not realize that collimator instructions were not being saved, and proceeded on the assumption that they were. There was no fail-safe to ensure the proper radiation was being administered.

Following the NYT article, the American Society for Radiation Oncology (ASTRO) issued a press release Feb. 3, 2010, committing itself to a six-point patient protection plan, the fifth being “Further developing our Integrating the Healthcare Enterprise—Radiation Oncology (IHE-RO) connectivity compliance program to ensure that medical technologies from different manufacturers can safely transfer information to reduce the chance of a medical error.”

Following the ASTRO press release, there were two patient safety meetings: 1) FDA Public Meeting (Mar. 30, 31, 2010) and 2) AAPM/ASTRO Safety Meeting (Jun. 9, 10 2010). Human error was considered the primary area where significant improvement was needed, followed by training and reducing device design complexity of use, including standardization and “human factors engineering” of these devices.

Most recently, the Technical Committee of ASTRO's IHE-RO produced a Framework titled “Quality Assurance with Plan Veto Profile” that aims to prevent the most serious errors as outlined in the NYT. Their goal is for independent QA vendors to provide this Quality Check role that would possess veto power over the treatment delivery in the event of a pending serious error, specifically excluding the Treatment Delivery Device vendors from this role. Complicating this Quality Assurance Plan Veto (QAPV per the IHE-RO Technical Committee) is the diversity of delivery machines and their operating systems that control beam delivery. Allowing a third party QA vendor to override a beam delivery, let alone designing for all these systems, is a daunting task facing many hurdles.

Systems for preventing unsafe beam operation already exist with most ionizing radiation treatment delivery devices (TDDs). It is available in the form of external safety interlocks provided by the institution. The most common is the door interlock with a simple electrical switch that is normally open when the door to the treatment room is open and closed when the door is closed. By way of example, a simplified schematic of a door interlock from a common LINAC system is illustrated with reference to FIG. 1. In addition to the door interlock, there may also be a visual warning indicator that the door to the treatment room is open. Notably, such systems have nothing to do with preventing pending radiation delivery to a patient based on some characteristics of that radiation delivery being improper and/or unsafe, of itself. Instead, these systems prevent delivery if circumstances exist which would result in hazards external to the patient treatment—e.g., the danger of a person walking into the treatment room during radiation delivery to a patient.

The entity operating the LINAC or other treatment delivery device is ultimately responsible for the safe use thereof, but conventionally these safety measures have focused on the many hazards associated with setup and use; for example, with the LINAC system, issues surrounding collision, beam portal imaging during treatment, beam limiters with certain accessories, and so on. The entity's assurance of safe treatment beyond hazard mitigation with safety features provided with RADONC equipment vendors (e.g., the door interlock) was limited to operator training of equipment use, visual and audio monitoring of the equipment. In particular, there have not been any means to automatically prevent unsafe treatments that help ensure an independent assessment of the pending treatment against the planned treatment.

### SUMMARY OF THE INVENTION

As can be seen from the foregoing, there has been for some time an urgent need for systems and methods that can prevent unsafe radiation delivery to patients. According to an embodiment of the present invention, a medical radiation delivery system comprises a treatment delivery device for delivering ionizing radiation to a patient, a treatment safety device including at least one of a warning indicator and an operational interlock for preventing improper operation of the treatment delivery device, and at least one processor and machine readable memory. The processor and memory are configured to execute a treatment delivery device control module for operating the treatment delivery device to deliver the ionizing radiation to the patient, and a treatment verification module for determining whether predetermined treatment verification criteria are met and operating the treatment safety device based thereon.

According to a method aspect, a method for preventing unsafe delivery of ionizing radiation to a patient comprises supplying at least one electronic verification input to a treatment verification module executed by at least one processor using machine readable code. Using the treatment verification module, it is determined whether the at least one electronic verification input satisfies at least one treatment verification criteria. Based on the determination of whether the at least one treatment verification criteria is satisfied, a verification output signal from the treatment verification module is generated, and a treatment safety device is operated based on the verification output signal to allow or inhibit the delivery of ionizing radiation by a treatment delivery device.

According to an advantageous aspect of the present invention, the treatment safety device includes an operational interlock that will prevent operation of the treatment delivery device. Preferably, the operational interlock is integrated into door interlock circuitry that will interrupt electrical power supply to the treatment delivery device.

These and other objects, aspects and advantages of the present invention will be better understood in view of the drawings and following detailed description of preferred embodiments.

### BRIEF DESCRIPTION OF THE DRAWINGS

FIG. 1 is a schematic diagram of a radiation oncology treatment system with a door interlock; and

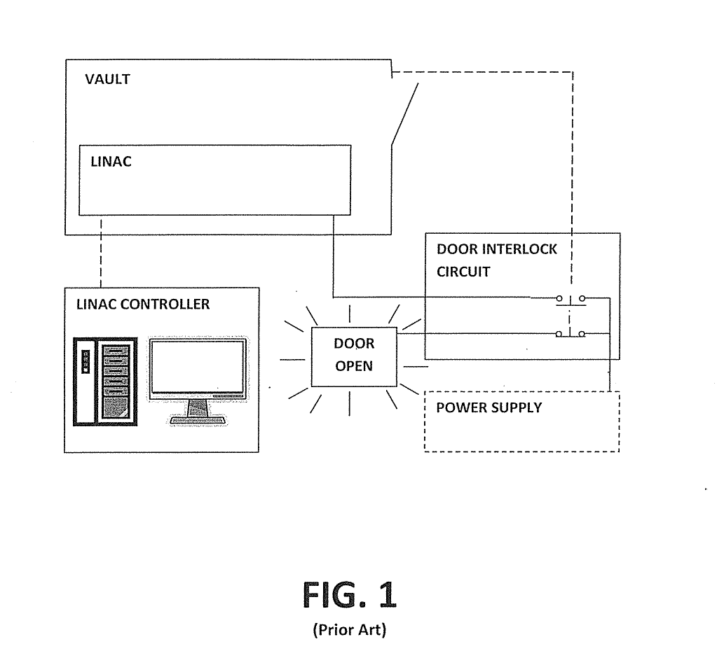

FIG. 2 is a schematic diagram of a radiation oncology treatment system, according to an embodiment of the present invention.

### DETAILED DESCRIPTION OF THE PREFERRED EMBODIMENTS

The present invention will now be described more fully hereinafter with reference to the accompanying drawings, in which a preferred embodiment of the invention is shown. This invention may, however, be embodied in many different forms and should not be construed as limited to the embodiments set forth herein. Rather, this embodiment is provided so that this disclosure will be thorough and complete, and will fully convey the scope of the invention to those skilled in the art.

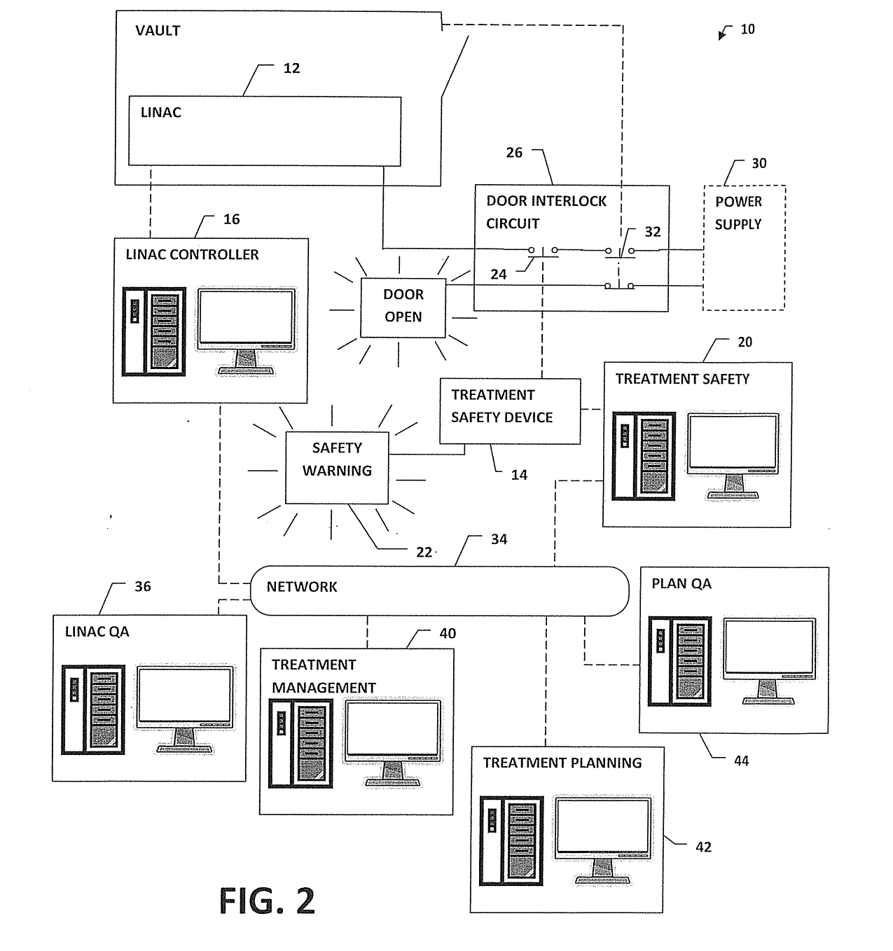

Referring to FIG. 2, according to an embodiment of the present invention, a radiation oncology system 10 is adapted to prevent unsafe delivery of radiation to a patient. As used herein, radiation oncology (RADONC) refers to the study and treatment of cancer disease with ionizing radiation. RADONC will ordinarily involve personnel, equipment and facilities designed for imaging and diagnosis of cancer patients, then cancer treatment planning and delivery to the patient. However, aspects of the present invention can readily be applied to medical delivery of radiation for other purposes (e.g., medical imaging unconnected with cancer), as well as to the safe administration of medical therapies involving potentially dangerous modalities other than ionizing radiation (e.g., magnetic resonance imaging (MRI), robotic surgery, etc.)

The radiation oncology system 10 includes a treatment delivery device (TDD) 12, a treatment safety device 14 and at least one processor and machine readable memory configured to execute a treatment delivery device control module 16 for operating the treatment delivery device and a treatment safety module 20. The treatment safety module 20 determines whether predetermined treatment verification criteria are met and, based on the determination, operates the treatment safety device 14 to prevent improper operation of the treatment delivery device 12. Preferably, the treatment safety device 14 activates at least one a warning indicator 22 and an operational interlock 24 to prevent improper operation.

The treatment delivery device 12 is a device for delivering a treatment to a patient. In the depicted embodiment, the treatment delivery device 12 is a device that uses ionizing radiation in a form that treats disease, and more particularly a linear accelerator (LINAC). As discussed above, a treatment delivery device may be used which does not deliver ionizing radiation, although the present invention is particularly advantageous in that context. In the context of treatment devices delivering ionizing radiation, there are teletherapy devices (radiation source external to the patient with an emitted beam of radiation directed toward the patient) and brachytherapy devices (radiation source internal to the patient with radiation directed toward the disease site). Examples of teletherapy devices are linear accelerators (LINAC) produced by Varian, Palo Alto, Calif.; TomoTherapy, Madison Wis.; Elekta, Crawley UK; Siemens, Erlangen, Germany; Accuray, Sunnyvale, Calif. Radiation isotopes such as Cobalt 60 are also used for teletherapy, produced by Viewray, Cleveland Ohio and formerly by Atomic Energy of Canada (AECL). Examples of brachytherapy devices are radioisotopes encapsulated in catheters and manipulated in a patient by a delivery system produced by Nucletron in Netherlands and Varian in Palo Alto Calif. Electronic X-ray sources are also encapsulated for catheter delivery, as exampled by Xoft, Sunnyvale Calif.

The treatment safety device 14 is a device that converts, as necessary, the logical determination of the treatment safety module 20 concerning treatment verification criteria into a form that can effect operation of the warning indicator 22 and/or operational interlock 22. The treatment safety device 14 is not necessarily a physically separate device from the computer(s) executing the linac control and/or treatment safety modules 20.

The warning indicator 22 is an indicator that is perceivable by an operator of the system 10. For example, the indicator 22 can incorporate visual, audible and/or tactile indicator elements. The warning indicator 22 could use a display associated with the linac control module 16 or some other system 10, and/or employ a special, purpose-built device, such as an illuminatable sign located in a control room for the treatment delivery device 16.

The operational interlock 24 is not necessarily limited to a particular interlock type. For example, the operation interlock could include a mechanical interlock and/or electrical interlock effect to prevent operation of the treatment delivery device 12 until the interlock is satisfied. Advantageously, the operational interlock 24 is an electrical interlock located in a circuit 26 to interrupt the supply of electrical power to the treatment delivery device 12 from a power supply 30. In the depicted embodiment, the circuit 26 is a door interlock circuit and the operational interlock 24 is a switch located therein, preferably in series with a door interlock switch 32, such that both switches must be closed for the treatment delivery device 12 to be operated. Alternately, where no existing interlock is in place, one could be added specifically for the system 10.

For clarity of illustration, the linac control module 16 and treatment safety module 20 are depicted as separate computers in communication via a network 24. While these modules operate independently of one another, the present invention does not necessarily require that they be run by separate computers, but could be executed by a single machine. Likewise, various functions of either module 16, 20 could be distributed across a plurality of computers. As used herein, a “computer” is an electronic device having at least one processor that can execute program instructions stored in machine readable memories to perform a function. The present invention is not necessarily limited to a particular number, type or configuration of computers and/or processors, or to a particular type or format of memory, or to any particular programming language.

A “network” generally includes the electronic components that allow two or more computers to communicate data therebetween. A wireless or wired local area network and the Internet are examples of networks, although the present invention is not necessarily limited thereto, and moreover, the term “network” can encompass multiple types and levels of networks used in conjunction to transfer data between computers. In the RADONC context, data transfer is advantageously through network connectivity using a NEMA DICOM standard (Digital Imaging Communication in Medicine). NEMA, the Association of Electrical and Medical Imaging Equipment Manufacturers, maintains the DICOM data standard is in use by most of the RADONC systems.

In operation of the system 10 as described above, when a pending treatment is received by the linac control module 16, the treatment safety module 20 operates the treatment safety device 14 such that the warning indicator 22 and/or operational interlock 24 will be activated (i.e., to prevent treatment) until the predetermined verification criteria are satisfied. Once the verification criteria are satisfied, the treatment safety device 14 releases the indicator 22 and/or interlock 24.

A plurality of verification criteria can be advantageously employed, either singularly or in various combinations, in connection with the present invention. The treatment safety module 20 can include a user interface for receiving entry of verification criteria, and acceptable data, ranges, thresholds, etc. that satisfy the criteria. In one version, satisfaction of the verification criteria can include ensuring a checklist procedure is followed, completed and signed off on by the therapists prior to treatment (i.e. ‘right patient, right site, right field size’). This checklist can be user-defined, and can include any setup, settings, or other visual checks the user may want but have no other way to require be done prior to treatment.

Via a user interface, a user can also enter general operating thresholds for the TDD 12 (e.g., thresholds that should generally not be exceeded for any use of the TDD, regardless of patient). If one or more such thresholds is not within the acceptable range, then the treatment verification criteria would not be satisfied, and the treatment safety device 14 will be operated to prevent treatment. A provision for operator override, to allow operation outside normal thresholds in special circumstances could also be provided—or in the face of any other verification criteria that is not satisfied.

External software applications can interact with the treatment safety module 20 as a further source of verification criteria. Examples of software modules include a treatment delivery device quality assurance (QA) module 36, a treatment management (TMS) module 40, a treatment planning (TPS) module 42 and a dose QA module 44. As with the control module 16 and treatment safety module 20, these additional modules can be executed by the same computer, or by different computers sharing data over the network 34. There can also be integration with third party devices that require beam control (i.e. motion tracking cameras that will turn the beam off if patient moves beyond a certain limit), and the like, and can also provide a back-up to a door interlock to prevent operation if the treatment room door is opened during treatment.

An example of how an input from treatment device QA (e.g., as received from the module 36) can be a treatment verification criteria in the RADONC context is if a specific energy on the TDD had a measured daily output that was unsafe.

Using all of the data available from the modules 40-44, the system 10 can effectively intercede to prevent unsafe treatment delivery from the TDD if the plan pending for delivery by the TDD is not the plan that was intended to be delivered to the patient. With respect to this distinction, it is useful to consider the relationship between an intended treatment and a pending treatment in the RADONC context.

The intended treatment is a TPS treatment plan after it has been approved by the RADONC personnel. A TPS treatment plan is pending in any state preceding treatment. There are several approval steps; the approval steps and criteria vary across institutions, depending upon the RADONC system. A first possible “approval step” is typically a review of several plan candidates at the TPS 42 console, resulting in selection of one, which then becomes the intended treatment. This selection may involve an independent plan computation check using the selected plan data, which can also be a verification criteria considered by the treatment safety module 20.

The transfer of the selected plan to the TMS 40 and then TDD control 16 and TDD 12 via the RADONC system's network 34 is a common need, and represents opportunity for data corruption, and if corruption occurs, the pending treatment is no longer the intended treatment. There is an opportunity for verification criteria to be utilized after transfer to the TMS 40; that is to compare the plan residing on the TMS 40 to the plan from the TPS 42, through the treatment safety module 20. Another similar verification criteria opportunity exists—comparing the plan residing on the TDD control module 16 after the transfer from TMS 40 using the treatment safety module 20. These two steps can help ensure that the pending treatment is equivalent to the intended treatment that was approved at the TPS 42.

Prior to the patient treatment, the Intended Treatment may be delivered to a dosimetry phantom, a QA measurement with the phantom being a surrogate patient. Following treatment, the dose delivered to the phantom (computed via the dose QA module 44) is compared to the planned dose associated with the intended treatment as another verification criteria; and if equivalent, then the Intended Treatment was delivered, confirming the TDD contains the approved plan.

A final verification criteria can involve the technician during the patient sessions, or fractions. Visual inspection of the patient setup and TDD parameters prior to treatment can prevent personnel errors, oversights, and incomplete processes before the pending treatment is delivered with more assurance that it is the intended treatment—this step can be accomplished via checklist procedure as described above.

There are other data sources, such as the log files following a treatment which provide the treatment data that was delivered. These data can also be captured and compared to the intended treatment data. This is after the fact, but for most RADONC treatments, the treatment course is over many fractions (e.g., 30 fractions) and the opportunity for verification after the pending treatment delivery was delivered and found to be incorrect still allows for the treatment safety module 20 to prevent delivery of the next fraction (typically the following day) via operation the treatment safety device 14.

The present invention makes is possible to evaluate a pending treatment against an intended treatment using the comparison of this data from these sources by the treatment safety module 20. As described above, in the RADONC context, the data transfer is preferably through network connectivity using a NEMA DICOM standard (Digital Imaging Communication in Medicine), which also allows communication with the linac control module 16, TMS 40 and TPS 42. For example, the treatment safety module 20 subscribes to the DICOM data traffic as a listener and captures intended and pending treatments when the data is pushed onto the network from the appropriate systems. The approved TPS treatment plan (intended treatment) data is available immediately after plan approval, and is stored by the treatment safety module 20 for later comparisons.

However, there is more than one method of treatment data transfer, depending on the RADONC system. The module 20 interprets the data, pending and intended, by means of software that compares the TDD 12 setup and characteristics to the TPS 42 plan. The TDD setup and characteristics may not be directly accessible by the treatment safety module 20, but can receive its setup instruction just prior to treatment from the TMS 40. The TDD controller 16 (controlling TDD/LINAC 12) may receive the TMS 40 instruction just prior to patient mode-up or it may recall it from its own database. The TDD controller 16 may be arranged to export its setup to the treatment safety module 20.

TMS (Treatment Management System—from the IHE-RO Technical Committee) is used as a descriptive acronym, although the planning to treatment interface (PTI) accomplished thereby can go by other names, such as Record and Verify (RV) (an early name), Oncology Information System (OIS) and Electronic Medical Record (EMR) which describe global departmental features. TMS incorporates multiple data points that can be used in the treatment safety module 20 determination, including the automatic setup parameters for the TDD 12, such as MLC positions, gantry angle, collimator position, beam energy, and beam monitor units. (An example of a TMS system that can work advantageously in connection with the current invention is the MOSAIQ system from Elekta.)

The present invention provides great flexibility and adaptability in preventing the delivery of an unsafe treatment from a TDD, and particularly in the case of the delivery of ionizing radiation. This allows entities that use such devices to automatically ensure that proper standards are being met, from an evaluation of tolerance values for a particular test that is monitored during a QA check to a treatment plan review of dose volume histograms (DVH) of anatomical structures (treatment targets and critical organs) as compared to an institution's standard of care.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the claims supported by this disclosure.

What is claimed is:

1. A medical radiation delivery system comprising:
a treatment delivery device for delivering ionizing radiation to a patient;
a treatment safety device including an operational interlock for preventing improper operation of the treatment delivery device, the operational interlock including an electrical interlock interrupting electrical power to the treatment delivery device; and
at least one processor and machine readable memory configured to execute:
a treatment delivery device control module for operating the treatment delivery device to deliver the ionizing radiation to the patient; and
a treatment verification module for determining whether predetermined treatment verification criteria are met and operating the treatment safety device based thereon.

2. The system of claim 1, further comprising an electrical door interlock for interrupting power to the treatment delivery device when a treatment room door is open, the electrical door interlock and the electrical interlock of the treatment safety device being located in series such that both must be closed to supply electrical power to the treatment delivery device.

3. The system of claim 1, wherein the treatment delivery device is a linear accelerator.

4. A medical radiation delivery system comprising:
a treatment delivery device for delivering ionizing radiation to a patient;
a treatment safety device including at least one of a warning indicator and an operational interlock for preventing improper operation of the treatment delivery device; and
at least one processor and machine readable memory configured to execute:
a treatment delivery device control module for operating the treatment delivery device to deliver the ionizing radiation to the patient; and
a treatment verification module for determining whether predetermined treatment verification criteria are met and operating the treatment safety device based thereon.

5. The system of claim 4, wherein the treatment delivery device control module and the treatment verification module are executed by separate processors.

6. The system of claim 4, wherein the treatment delivery device is a linear accelerator.

7. The system of claim 4, wherein the treatment safety device includes an operational interlock preventing the operation of the treatment delivery device until the interlock is satisfied.

8. The system of claim 7, wherein the operational interlock is an electrical interlock interrupting electrical power to the treatment delivery device.

9. The system of claim 8, further comprising an electrical door interlock for interrupting power to the treatment delivery device when a treatment room door is open, the electrical door interlock and the operational interlock being located in series.

10. The system of claim 4, wherein the treatment verification criteria include a satisfactory comparison between at least one element of a pending treatment plan communicated to the treatment delivery device control module for execution by the treatment delivery device and at least one element of an intended treatment plan.

11. The system of claim 4, wherein the treatment verification criteria include a satisfactory comparison between at least one element of a pending treatment plan communicated to the treatment delivery device control module for execution by the treatment delivery device and at least one predetermined operating threshold of the treatment delivery device.

12. The system of claim 11, wherein the treatment verification module includes a user interface for receiving entry of the at least one predetermined operating threshold.

13. The system of claim 4, wherein the treatment verification criteria include an electronic indication of completion of a pre-treatment checklist.

14. The system of claim 4, wherein the treatment verification criteria include a satisfactory comparison of a previously delivered treatment fraction with an intended treatment plan.

15. The system of claim 4, wherein the treatment verification criteria include quality assurance data from a quality assurance check on the treatment delivery device falling within a predetermined threshold.

16. The system of claim 4, wherein the treatment verification criteria include quality assurance data from a quality assurance evaluation of an intended treatment plan falling within a predetermined threshold.

17. A method for preventing unsafe delivery of ionizing radiation to a patient, the method comprising:
supplying at least one electronic verification input to a treatment verification module executed by at least one processor using machine readable code;
determining, using the treatment verification module, whether the at least one electronic verification input satisfies at least one treatment verification criteria;
generating, based on the determination of whether the at least one treatment verification criteria is satisfied, a verification output signal from the treatment verification module; and
operating a treatment safety device based on the verification output signal to allow or inhibit the delivery of ionizing radiation by a treatment delivery device.

18. The method of claim 17, wherein the at least one electronic verification input includes at least one element of an pending treatment plan for execution by the treatment delivery device and at least one element of an intended treatment plan for the patient.

19. The method of claim 17, wherein the at least one electronic verification input includes at least one element of a pending treatment plan for execution by the treatment delivery device and at least one predetermined operating threshold of the treatment delivery device.

20. The method of claim 17, wherein the at least one electronic verification input includes an indication of completion of a pre-treatment checklist.

21. The method of claim 17, wherein the at least one electronic verification input includes information on a previously delivered treatment fraction and a corresponding intended treatment fraction.

22. The method of claim 17, wherein the at least one electronic verification input includes quality assurance data from a quality assurance check on the treatment delivery device.

23. The method of claim 17, wherein the at least one electronic verification input includes include quality assurance data from a quality assurance evaluation of an intended treatment plan.

24. The method of claim 17, wherein operating the treatment safety device based on the verification output signal includes operating a warning indicator detectable by an operator of the treatment delivery device.

25. The method of claim 17, wherein operating the treatment safety device based on the verification output signal includes operating an operational interlock.

26. The method of claim 25, wherein operating an operational interlock includes selectively interrupting an electrical power supply to the treatment delivery device.

27. The method of claim 26, wherein selectively interrupting an electrical power supply to the treatment delivery device includes selectively interrupting the electrical power supply in the same circuit as a door interlock.
